(19) United States
(12) Patent Application Publication
(10) Pub. No.: US 2025/0059301 A1
(43) Pub. Date: Feb. 20, 2025

(54) Fusion Protein Construct

(71) Applicant: NORTHWESTERN UNIVERSITY, Evanston, IL (US)

(72) Inventors: Milan Mrksich, Hinsdale, IL (US); Justin A. Modica, Chicago, IL (US)

(21) Appl. No.: 18/805,059

(22) Filed: Aug. 14, 2024

Related U.S. Application Data
(63) Continuation of application No. 16/307,621, filed on Dec. 6, 2018, now Pat. No. 12,098,215.
(60) Provisional application No. 62/346,369, filed on Jun. 6, 2016.

(51) Int. Cl. C07K 16/46; A61K 39/00; A61P 19/02; A61P 35/00; A61P 37/00; C07K 16/32; C12N 9/18

(52) U.S. Cl. CPC C07K 16/46; A61P 19/02; A61P 35/00; A61P 37/00; C07K 16/32; C12N 9/18; C12Y 301/01074; A61K 2039/505; C07K 2317/24; C07K 2317/55; C07K 2317/565; C07K 2317/73; C07K 2317/92; C07K 2317/94; C07K 2319/00; C07K 2319/61; C12Y 201/01037; C12Y 301/21002; C12Y 302/01021; C12Y 304/11018; C12Y 304/24024; C12Y 305/02006

(57) ABSTRACT

The disclosure provides constructs comprising a first fusion protein, a second fusion protein, and a linker, wherein the first fusion protein and the second fusion protein each include an affinity reagent and a reactive enzyme, and the linker includes a first and second functional groups specific for irreversibly inhibiting the first and second fusion protein reactive enzymes. The disclosure further provides a method including (a) contacting a first fusion protein including an affinity reagent and a reactive enzyme with a linker including a functional group specific for irreversibly inhibiting the first fusion protein reactive enzyme thereby coupling the first fusion protein and the linker, and (b) contacting a second fusion protein including an affinity reagent and a reactive enzyme with the linker, the linker including a functional group specific for irreversibly inhibiting the second fusion protein reactive enzyme thereby coupling the second fusion protein and the linker.

### FIELD OF THE DISCLOSURE

The disclosure relates generally to fusion protein constructs and their methods of preparation and use. More particularly, the disclosure relates to precisely-defined fusion protein constructs comprising at least a first fusion protein and a second fusion protein coupled by a linker, prepared by a modular synthesis.

### SUBMISSION OF SEQUENCE LISTING

The content of the following Sequence Listing XML is incorporated herein by reference in its entirety: Filename: 2016-048_SeqListing.xml; Size: 35,069 bytes; Created: Nov. 7, 2024.

### BACKGROUND

Biologic drugs, specifically monoclonal antibodies (mAbs) and those that incorporate antibody fragments are of interest in the pharmaceutical industry due to their ability to specifically target disease-related proteins in circulation or on cell surfaces. Next-generation antibody therapeutics, those that show enhanced therapeutic potential, have been created by increasing the antibody valency, attaching effector molecules that increase cytotoxicity and half-life, and by using formats that direct the immune system toward clearing targets. These molecules, however, are often challenging to produce in high-yield and with synthetic flexibility that permits a desired function to be systematically optimized.

For example, current antibody formats with expanded functionality must be arduously optimized for stability or function as they feature multiple domains that must be expressed concurrently. Current chemical methods require the engineering of a reactive amino acid or the use of a preexisting reactive amino acid side chain to attach payloads to the antibody scaffold. In many cases, products of these reactions yield heterogeneous populations of products. Such direct amino-acid modifications may also not be stable or cause deleterious effects to the pharmacological properties of the parent molecule (e.g. increased hydrophobicity, aggregation, immunogenicity, etc.) therefore a great deal of effort must be directed toward choosing a suitable location for attaching the payload.

Additionally, current antibody technologies do not enable the product of scaffolds where the geometry (e.g., cyclic molecules, molecules with variable distances between domains, etc), orientation, stoichiometry, and valency can be systematically controlled outside that of what is enabled by classical polypeptide engineering. This restriction of production technologies places an enormous constraint on the therapeutic space that current formats can investigate. Current protein engineering methods are limited to the intrinsic repertoire of natural polypeptide folds/peptide linkers to achieve a desired construct. Therefore, non-natural formats that may exhibit greater efficacy cannot be prepared using conventional engineering methods. Further, due to toxicity constraints or the metabolic profiles of the expression hosts, some effector molecules cannot be effectively produced together in a one culture system. Additionally, correct folding, assembly, and purification of larger molecules becomes more difficult as the complexity of the molecule is increased, thereby decreasing yield and increasing cost.

Further, industrial production of antibody molecules generally requires expression of antibodies or antibody-like fragments in eukaryotic hosts, which are less robust than prokaryotic hosts and have reduced product yields compared to prokaryotic hosts, resulting in higher operating costs relative to antibodies or antibody-like fragments that can be expressed in prokaryotic hosts.

Thus, there exists a need in the art for a cost effective method of preparing next generation antibody therapeutics with high molecular weights that allows for the creation of a diverse library of antibody-like drugs with altered valency, geometry, effector function and stoichiometry simply by the combination of a comparatively small number of building blocks.

### SUMMARY

One aspect of the disclosure provides constructs comprising a first fusion protein, a second fusion protein, and a linker, wherein the first fusion protein and the second fusion protein each include an affinity reagent and a reactive enzyme, and the linker includes a first functional group specific for irreversibly inhibiting the first fusion protein reactive enzyme at a first terminus, and a second functional group specific for irreversibly inhibiting the second fusion protein reactive enzyme at a second terminus.

Another aspect of the disclosure provides a method including (a) contacting a first fusion protein including an affinity reagent and a reactive enzyme with a linker including a functional group specific for irreversibly inhibiting the first fusion protein reactive enzyme at a first terminus thereby coupling the first fusion protein and the linker at the first terminus, and (b) contacting a second fusion protein including an affinity reagent and a reactive enzyme with a second terminus of the linker, the second terminus of the linker including a functional group specific for irreversibly inhibiting the second fusion protein reactive enzyme thereby coupling the second fusion protein and the linker at the second terminus.

Another aspect of the disclosure provides a construct prepared by the method of the disclosure.

Another aspect of the disclosure provides a method comprising administering a construct of the disclosure to a patient in need thereof. Other aspects of the disclosure provide uses of the construct of the disclosure as a medicament and/or as a diagnostic.

Further aspects and advantages will be apparent to those of ordinary skill in the art from a review of the following detailed description. While the constructs and their methods of making and use are susceptible of embodiments in various forms, the description hereafter includes specific embodiments with the understanding that the disclosure is illustrative, and is not intended to limit the invention to the specific embodiments described herein.

### BRIEF DESCRIPTION OF THE DRAWINGS

FIG. 1 shows Trastuzumab F(ab) (TFab) fusion gene/protein constructs.

FIG. 2 shows the SDS-PAGE gel of purified TFab-cutinase and TFab-SnapTag fusions.

FIG. 3 shows the SDS-PAGE gel of bivalent constructs.

FIG. 4 shows cell viability assay data of trastuzumab and a fusion protein construct, B-13.

FIG. 5 is a plot of serum concentration of the B-13 construct in mice over time.

FIG. 6 shows the results of the effect of B-13 adminstration on mice having BT474 tumor xenografts. A) Tumor volume progression of the animals treated over 4 weeks with vehicle, B13 at 2.5 and 5 mg/kg daily, and doxorubicin (dox) twice a week at 2.5 mg/kg. At day 28, there is a significant difference in tumor volume between vehicle vs. doxorubicin (p=0.0002) and vehicle vs. the two B13 treatment groups (p=0.0001). B) Total body weight measurement of the animals in the four treatment cohorts. There is a significant difference in total body weight between vehicle and doxorubicin (p=0.0116) but no significant difference between vehicle and B13 treatment at 2.5 mg/kg (p=0.2237) or 5 mg/kg (p=0.4297) C) Organ weight of the animals in the four treatment cohorts. There is no significant difference in organ weight among the groups (p=0.9493). P-values were calculated using a Two Way ANOVA, in Graph Pad 7.0.

### DETAILED DESCRIPTION

Provided herein are constructs and methods of making and using the constructs. The constructs comprise a first fusion protein, a second fusion protein, and a linker, wherein the first fusion protein and the second fusion protein each include an affinity reagent and a reactive enzyme, and the linker includes a first functional group specific for irreversibly inhibiting the first fusion protein reactive enzyme at a first terminus, and a second functional group specific for irreversibly inhibiting the second fusion protein reactive enzyme at a second terminus. In embodiments, the first fusion protein reactive enzyme and the second fusion protein reactive enzyme are different. In embodiments, the first fusion protein reactive enzyme and the second fusion protein reactive enzyme are the same. In embodiments, the first fusion protein affinity reagent and the second fusion protein affinity reagent are different. In embodiments, the first fusion protein affinity reagent and the second fusion protein affinity reagent are the same. Optionally, in embodiments, each affinity reagent is independently selected from the group consisting of antibody or fragment thereof, small molecule, monobody, protein, and combinations thereof.

### Constructs of Fusion Proteins of Affinity Reagent and Reactive Enzymes

The disclosed constructs comprise fusion proteins of an affinity reagent and a reactive enzyme linked via a linker.

As used herein and unless specified otherwise, “affinity reagent” refers to a moiety that exhibits affinity for a desired target, for example, the ability to bind to a target epitope in a therapeutic system and/or bind or recognize an analyte in a biosensor system. Examples of affinity reagents include, but are not limited to, antibodies or fragments thereof, small molecules, monobodies, and proteins.

As used herein and unless specified otherwise, “reactive enzyme” refers to an enzyme comprising an active-site residue that can couple to a functional group specific for irreversibly inhibiting the active-site residue.

As used herein and unless specified otherwise, “protein” refers to naturally occurring polypeptides, polypeptides comprising natural and/or unnatural amino acids, proteins in phage, therapeutic proteins, and antibody domains either synthetically or naturally derived.

As used herein a “polypeptide” refers to a polymer comprised of amino acid residues. Polypeptides are understood in the art and include without limitation an antibody, an enzyme, a structural polypeptide, and a hormone. Polypeptides of the present disclosure may be either naturally occurring or non-naturally occurring.

Naturally occurring polypeptides include without limitation biologically active polypeptides (including antibodies) that exist in nature or can be produced in a form that is found in nature by, for example, chemical synthesis or recombinant expression techniques. Naturally occurring polypeptides also include lipoproteins and post-translationally modified proteins, such as, for example and without limitation, glycosylated proteins.

Antibodies contemplated for use in the methods and compositions of the present disclosure include without limitation antibodies that recognize and associate with a target molecule either in vivo or in vitro.

Structural polypeptides contemplated by the disclosure include without limitation actin, tubulin, collagen, elastin, myosin, kinesin and dynein.

Non-naturally occurring polypeptides contemplated by the present disclosure include but are not limited to synthetic polypeptides, as well as fragments, analogs and variants of naturally occurring or non-naturally occurring polypeptides as defined herein. Non-naturally occurring polypeptides also include proteins or protein substances that have D-amino acids, modified, derivatized, or non-naturally occurring amino acids in the D- or L-configuration and/or peptidomimetic units as part of their structure. The term “protein” typically refers to large polypeptides. The term “peptide” typically refers to short polypeptides.

Non-naturally occurring polypeptides are prepared, for example, using an automated polypeptide synthesizer or, alternatively, using recombinant expression techniques using a modified polynucleotide which encodes the desired polypeptide.

As used herein a “fragment” of a polypeptide is meant to refer to any portion of a polypeptide or protein smaller than the full-length polypeptide or protein expression product.

As used herein an “analog” refers to any of two or more polypeptides substantially similar in structure and having the same biological activity, but can have varying degrees of activity, to either the entire molecule, or to a fragment thereof. Analogs differ in the composition of their amino acid sequences based on one or more mutations involving substitution, deletion, insertion and/or addition of one or more amino acids for other amino acids. Substitutions can be conservative or non-conservative based on the physico-chemical or functional relatedness of the amino acid that is being replaced and the amino acid replacing it.

As used herein a “variant” refers to a polypeptide, protein or analog thereof that is modified to comprise additional chemical moieties not normally a part of the molecule. Such moieties may modulate, for example and without limitation, the molecule's solubility, absorption, and/or biological half-life. Moieties capable of mediating such effects are disclosed in Remington's Pharmaceutical Sciences (1980). Procedures for coupling such moieties to a molecule are well known in the art.

Antibodies and fragments or derivatives thereof, including but not limited to Fab′ fragments, F(ab)2 fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art are contemplated.

In embodiments of the foregoing aspect, at least one antibody or fragment thereof of the construct includes trastuzumab or fragment thereof, raxibacamab or fragment thereof, tocilizumab or fragment thereof, brentuximab or fragment thereof, ofatumumab or fragment thereof, belimumab or fragment thereof, ramucirumab or fragment thereof, vedolizumab or fragment thereof, obinutuzumab or fragment thereof, pembrolizumab or fragment thereof, ranibizumab or fragment thereof, pertuzumab or fragment thereof, denosumab or fragment thereof, catumaxomab or fragment thereof, golimumab or fragment thereof, siltuximab or fragment thereof, natalizumab or fragment thereof, panitumumab or fragment thereof, denosumab or fragment thereof, and combinations of the foregoing.

In embodiments, the linker is a polyoxazoline, polyacrylomorpholine, polyvinylpyrrolidone, polyphosphazene, polyethylene-co-maleic acid anhydride, polystyrene-co-maleic acid anhydride, poly(1-hydroxymethylethylene hydroxymethyl formal) (“PHF”), a polyhydroxyalkylacrylate, 2-methyacryloyloxy-2′-ethyltrimethylammonium phosphate (“MPC”), or a structure selected from:

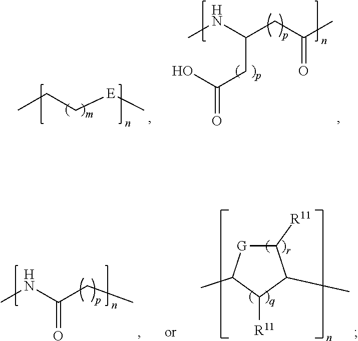

wherein: m is 0-10; n is 1-100; each p independently is 0, 1, 2, 3, or 4; q is 0, 1, or 2; r is 1 or 2; E is NH or CHR¹⁰; G is O, CH₂, CHOH, CHNH₂, CHCOOH, or CHSO₃H; R¹⁰ is OH, NH₂, or COOH; and each R¹¹ independently is H, OH, NH₂, or COOH.

In embodiments, each reactive enzyme is independently selected from the group consisting of cutinase, SnapTag, HaloTag, relaxase domains of type I DNA topoisomerases, beta-lactamase, glycosidase, matrix metalloproteinase, cytoplasmic protein tyrosine kinase domains, alkaline phosphatases, protein-tyrosine-phsophatases, mutants of 23S rRNA (adenine (2503)-C(2))-methyltransferases, glucosidases, N-6 adenine-specific DNA methylase, N(4)-cytosine-specific DNA methylase, DNA (cytosine-5-)-methyltransferase, mutants of haloalkane dehalogenases, HNH endonucleases, nicking endonucleases, gelatinases B, gelatinases A, stromelysins, fatty acid amide hydrolases, esterases, cytochrome P450s, methionine aminopeptidases, and combinations thereof. In embodiments, each reactive enzyme is independently selected from the group consisting of cutinase, the haloalkane dehydrogenase HaloTag, the mutant O⁶-alkylguanine DNA alkyltransferase, SnapTag, relaxase domains of type I DNA topoisomerases, betalactamase, glycosidase, matrix metalloproteinase, and combinations thereof. In embodiments, each reactive enzyme is independently selected from the group consisting of cutinase, the haloalkane dehydrogenase HaloTag, the mutant O⁶-alkylguanine DNA alkyltransferase, SnapTag, and combinations thereof.

In embodiments, the first fusion protein reactive enzyme and the second fusion protein reactive enzyme are different. In embodiments, the first fusion protein reactive enzyme and the second fusion protein reactive enzyme are the same. In embodiments, the first fusion protein affinity reagent and the second fusion protein affinity reagent are different. In embodiments, the first fusion protein affinity reagent and the second fusion protein affinity reagent are the same. Optionally, in embodiments, each affinity reagent is independently selected from the group consisting of antibody or fragment thereof, small molecule, monobody, protein, and combinations thereof.

In embodiments, the fusion proteins of the affinity reagent and the reactive enzyme are formed by conventional synthetic chemistry techniques to attach the reactive enzyme to the affinity reagent. In some cases, where the affinity reagent comprises a protein or antibody, a fusion protein is produced by expressing a polynucleotide sequence that encodes the affinity reagent and the reactive enzyme. In some aspects, the disclosure provides a vector comprising the polynucleotide operably linked to a promoter. In further aspects, the disclosure provides a host cell comprising the vector. In some embodiments, the host cell is an Escherichia coli cell. In further embodiments, the host cell is a mammalian cell. In related embodiments, the host cell is a Chinese Hamster Ovary (CHO) cell. In still further embodiments, the host cell is a yeast cell. In further aspects, a method of producing a fusion protein disclosed herein is provided, the method comprising the step of culturing a host cell of the disclosure under conditions appropriate to induce expression of the polypeptide. In related embodiments, the polypeptide is isolated.

### Affinity Reagents

The constructs of the disclosure include fusion proteins which comprise an affinity reagent that provides functionality to the fusion protein. Thus, a construct comprising a first fusion protein and a second fusion protein having precisely defined structures and functions can be assembled. Examples of functionality imparted to the fusion protein through the affinity reagent include, but are not limited to, enhanced targeting of disease-related proteins in circulation or on cell surfaces, increased cytotoxicity, immunomodulation, drug delivery, use as contrast agents, use as diagnostic agents, or combinations of the foregoing.

The first affinity reagent and the second affinity reagent can be the same or different. Each affinity reagent can be independently selected from the group consisting of antibody or fragment thereof, small molecule, monobody, protein, and combinations thereof.

In embodiments, the affinity reagent comprises an antibody or fragment thereof. The antibody or fragment thereof can be selected from the group consisting of a light chain variable domain (V_{L}), a light chain constant domain (C_{L}), a heavy chain variable domain (V_{H}), a heavy chain constant domain (C_{H}1), and a combination thereof. The antibody or fragment thereof can be a chimeric antibody, a human antibody, and a humanized antibody. Examples of antibodies or fragments thereof include, but are not limited to, trastuzumab or fragment thereof, raxibacamab or fragment thereof, tocilizumab or fragment thereof, brentuximab or fragment thereof, ofatumumab or fragment thereof, belimumab or fragment thereof, ramucirumab or fragment thereof, vedolizumab or fragment thereof, obinutuzumab or fragment thereof, pembrolizumab or fragment thereof, ranibizumab or fragment thereof, pertuzumab or fragment thereof, denosumab or fragment thereof, catumaxomab or fragment thereof, golimumab or fragment thereof, siltuximab or fragment thereof, natalizumab or fragment thereof, panitumumab or fragment thereof, and denosumab or fragment thereof.

In embodiments, the affinity reagent comprises a small molecule. The small molecule can be a drug. Examples of small molecule drugs include, but are not limited to, aldesleukin, alendronic acid, alfaferone, alitretinoin, allopurinol, aloprim, aloxi, altretamine, aminoglutethimide, L-asparaginase, amifostine, amrubicin, amsacrine, anastrozole, anzmet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulphate, broxuridine, bortezomib, bleomycin, busulfan, calcitonin, campath, capecitabine, carboplatin, carmustine, casodex, cefesone, celmoleukin, cerubidin, chlorambucil, cisplatin, colaspase, cladribin, clodronic acid, cyclophosphamide, cytarabine, dacarbazine, dactinomycin, daunoxome, decadron, decadron phosphate, delestrogen, denileukin diftitox, depomedrol, deslorelin, dexrazoxane, daunorubicin, diethylstilbestrol, 2′,2′-difluorodeoxycytidine, diflucan, docetaxel, doxifluridine, doxorubicin, dronabinol, DW-166HC, eligard, elitek, ellence, emend, epirubicin, epoetin-alfa, epogen, eptaplatin, ergamisol, estrace, estradiol, estramustine sodium phosphate, ethinylestradiol, ethyol, etidronic acid, etopophos, etoposide, fadrozole, farstone, filgrastim, finasteride, fligrastim, floxuridine, fluconazole, fludarabin, fludarabin phosphate, 5-fluorodeoxyuridine, 5-fluorodeoxyuridine monophosphate, 5-fluorouracil (5-FU), fluoxymesterone, flutamide, hexamethylmelamine, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, hydroxyprogesterone caproate, ibritumomab tiuxetan, idarubicin, ifosfamide, interferon-alpha, interferon-alpha-2,interferon-alpha-2α, interferon-alpha-2β, interferon-alpha-n1, interferon-alpha-n3, interferon-beta, interferon-gamma-1α, interleukin-2, intron A, iressa, irinotecan, kytril, lentinan sulphate, letrozole, leucovorin, leuprolide, leuprolide acetate, levamisole, levofolic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, N-phosphonoacetyl L-aspartate (PALA), picibanil, pilocarpine hydrochloride, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofiran, sobuzoxane, solu-medrol, streptozocin, strontium-89 chloride, Synthroid, tamoxifen, tamsulosin, tasonermin, tastolactone, taxoter, teceleukin, temozolomide, teniposide, testosterone propionate, testred, thioguanine, thiotepa, thyrotropin, tiludronic acid, topotecan, toremifen, tositumomab, tastuzumab, teosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin-stimalamer, zofran; ABI-007, acolbifen, actimmune, affinitak, aminopterin, arzoxifen, asoprisnil, atamestane, atrasentan, avastin, BAY 43-9006 (sorafenib), CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon-gamma, intron-PEG, ixabepilone, keyhole limpet hemocyanine, L-651582, lanreotide, lasofoxifen, libra, lonafarnib, miproxifen, minodronate, MS-209, liposomal MTP-PE, MX-6, nafarelin, nemorubicin, neovastat, nolatrexed, oblimersen, onko-TCS, osidem, paclitaxel polyglutamate, pamidronate disodium, PN-401, QS-21, quazepam, R-1549, raloxifen, ranpirnas, 13-cis-retic acid, satraplatin, seocalcitol, T-138067, tarceva, taxoprexin, thymosin-alpha-1, tiazofurin, tipifarnib, tirapazamine, TLK-286, toremifen, transMID-107R, valspodar, vapreotide, vatalanib, verteporfin, vinflunin, Z-100, zoledronic acid and combinations of the foregoing.

In embodiments, the affinity reagent comprises a protein. In embodiments, the protein is selected from the group consisting of a natural protein, a natural amino acid, an unnatural amino acid, a protein in phage, a therapeutic protein, a synthetic antibody domain, and combinations of the foregoing. In embodiments, the protein is selected from the group consisting of an unnatural amino acid, a protein in phage, a therapeutic protein, a synthetic antibody domain, and combinations of the foregoing.

In embodiments, the affinity reagent is selected from the group consisting of designed ankyrin repeat proteins (DARPins), HEL4 Vh domains (Predator), Z-domain of staphylococcal protein A (Affibody), archeal “7 kDa DNA binder” protein family (Affitin), carbohydrate-binding module (CBD domain), cystine-knot miniprotein (knottin), fibronectin type III domain (monobody, Adnectin), γ-B-crystallin (Affilin), cystatins (Affimers), triple helix coiled coil domains (Alhabodies), lipocalin domains (Anticalins), A domains of various membrane receptors (Avimers), SH3 domains of Fyn (Fynomers), Kunitz domain peptides, and combinations thereof.

### Reactive Enzyme

The fusion proteins of the disclosure include a reactive enzyme that can couple to a functional group specific for irreversibly inhibiting the reactive enzyme that is present on a linker. Thus, a construct comprising a first fusion protein and a second fusion protein having precisely defined structures can be assembled using a modular approach using synthetic linkers that selectively couple to an active-site residue in the fusion protein reactive enzymes.

The first fusion protein reactive enzyme and the second fusion protein reactive enzyme can be the same or different. Each reactive enzyme can be independently selected from the group consisting of cutinase, SnapTag, HaloTag, relaxase domains of type I DNA topoisomerases, beta-lactamase, glycosidase, matrix metalloproteinase, cytoplasmic protein tyrosine kinase domains, alkaline phosphatases, protein-tyrosine-phsophatases, mutants of 23S rRNA (adenine (2503)-C(2))-methyltransferases, glucosidases, N-6 adenine-specific DNA methylase, N(4)-cytosine-specific DNA methylase, DNA (cytosine-5-)-methyltransferase, mutants of haloalkane dehalogenases, HNH endonucleases, nicking endonucleases, gelatinases B, gelatinases A, stromelysins, fatty acid amide hydrolases, esterases, cytochrome P450s, methionine aminopeptidases, and combinations thereof.

In embodiments, each reactive enzyme is independently selected from the group consisting of cutinase, HaloTag, SnapTag, relaxase domains of type I DNA topoisomerases, betalactamase, glycosidase, matrix metalloproteinase, and combinations thereof. In embodiments, each reactive enzyme is independently selected from the group consisting of cutinase, HaloTag, SnapTag, and combinations thereof.

In embodiments, the reactive enzyme is a mobility class (MOB) relaxase domain of type I DNA topoisomerases. The MOB relaxase domain of type-I DNA topoisomerases are enzymes that attach covalently to small cognate origin of transfer (oriT) DNA sequences located within a mobility plasmid that also encodes the relaxase protein. Such enzymes cleave the oriT DNA sequence at a specific nucleotide residue and covalently attach to the oriT DNA sequence at the point of cleavage. MOB relaxase domain of type I DNA topoisomerases can be characterized by family, wherein each family member has a similar structure and mechanism. Examples of known MOB relaxase domains of type-I DNA topoisomerase families include MOB_{F}, MOB_{H}, MOB_{Q}, MOB_{C}, MOB_{P}, and MOB_{V}. OriT sequences for members of each relaxase family are specific to the mobility plasmids from which each relaxase protein sequence is derived. Known examples of relaxase families and their associated plasmids include, but are not limited to, MOB_{F} family: pF, pR100, pR388, pWWO, pMFLV02, pREB5, pREC1, and pNAC3; MOB_{H} family: pR27, pCAR1, pSXT, pIP1202, pMOL28, and pPNAP01; MOB_{C} family: pCloDF13, p23023, pCRY, pAD1, pSKU146.2, pLM7, and pSt0; MOB_{Q} family: pRSF1010, pPRO2, pTi, pAt, pGOX3, pWCFS103, pBM300, pCAUL01, pTB3, pKJ50, and pRF; MOB_{P} family: pRP4, pEST4011, pBI1063, pR64, pET46, pACRY04, pRAS3, pVirD2pTi, pColE1, pRA3, pVT745, pCP13, pFMC, pRP4, pR64, pK, pRAY, pCH4, pET35, pNP40, pCF10, pVir, pBOT3502, pMD136, pCIZ2, pS194, pSK639, and pRJ9; and MOB_{V} family: pMV158, pE194, pUB110, pBBR1, pTn5520, pTn4555, pIncU, and pIncX, wherein the subscrip “p” stands for plasmid.

### Linkers

The linker includes at least a first terminus and a second terminus. The linker further includes at the first terminus, a first functional group specific for irreversibly inhibiting a fusion protein. The linker further includes at the second terminus, a second functional group specific for irreversibly inhibiting a fusion protein.

In embodiments, the linker further comprises a nitrogen-containing C₃₋₇heterocycle, polyoxazoline, polyacrylomorpholine, polyvinylpyrrolidone, polyphosphazene, polyethylene-co-maleic acid anhydride, polystyrene-co-maleic acid anhydride, poly(1-hydroxymethylethylene hydroxymethyl formal) (“PHF”), a polyhydroxyalkylacrylate (e.g., 2-hydroxyethylmethacrylate), 2-methyacryloyloxy-2′-ethyltrimethylammonium phosphate (“MPC”), or a combination thereof. For example, the linker can include polyoxazoline, polyacrylomorpholine, polyvinylpyrrolidone, polyphosphazene, polyethylene-co-maleic acid anhydride, polystyrene-co-maleic acid anhydride, PHF, a polyhydroxyalkylacrylate, or MPC.

In embodiments, the linker comprises a group selected from

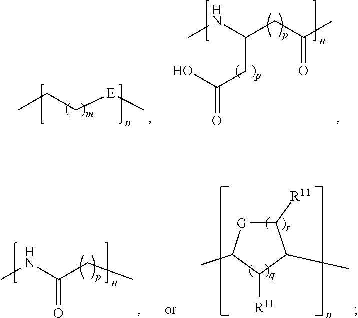

wherein:
m is 0-10;
n is 1-100;
each p independently is 0, 1, 2, 3, or 4;
q is 0, 1, or 2;
r is 1 or 2;

### E is O, NH or CHR¹⁰;

G is O, CH₂, CHOH, CHNH₂, CHCOOH, or CHSO₃H;
R¹⁰ is OH, NH₂, or COOH; and
each R¹¹ independently is H, OH, NH₂, or COOH.

In embodiments, the linker comprises

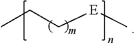

In some embodiments, E is O. In various embodiments, E is NH. In some embodiments, E is CHR¹⁰. For example E can include CHOH, CHNH₂, CHCOOH, or CHSO₃H. In these embodiments, m can be 0, 1, 2, 3, 4, 5, 6, 7, 8, 9, or 10. For example, m can be 1, 2, 3, 4, or 5. In some embodiments, m is 1, 2, or 3. In various embodiments, n can be 1-50, 1-40, 1-30, 1-20, 1-25, 1-20, 1-15, 1-10, or 1-5.

In embodiments, the linker comprises

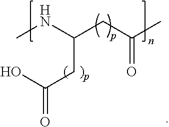

In some cases, p is 1, 2, or 3 (e.g., 1 or 2). For example, the linker can include aspartic acid, glutamic acid, or gamma-glutamic acid. In various embodiments, n can be 1-50, 1-40, 1-30, 1-20, 1-25, 1-20, 1-15, 1-10, or 1-5.

In embodiments, the linker comprises

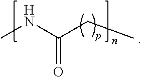

In some cases, p is 0, 1, 2, or 3 (e.g., 1 or 2). In various embodiments, n can be 1-50, 1-40, 1-30, 1-20, 1-25, 1-20, 1-15, 1-10, or 1-5.

In embodiments, the linker comprises

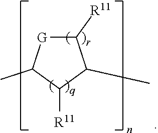

In embodiments, G is O. In embodiments wherein G is O, each R¹¹ can independently be H or OH. In embodiments, G is CH₂. In embodiments wherein G is CH₂, each R¹¹ can independently be H or OH, provided at least one R¹¹ is OH. In embodiments each R¹¹ is OH. In various embodiments wherein G is CH₂, each R¹¹ can independently be H or NH₂, provided at least one R¹¹ is NH₂. In various embodiments wherein G is CH₂, each R¹¹ can independently be H or COOH, provided at least one R¹¹ is COOH. In embodiments, G is CHOH. In embodiments wherein G is CHOH, each R¹¹ can independently be H or OH. In embodiments, each R¹¹ is OH. In embodiments, G is CHNH₂. In embodiments wherein G is CHNH₂, each R¹¹ can independently be H or NH₂. In embodiments, G is CHCOOH. In embodiments wherein G is CHCOOH, each R¹¹ can independently be H or COOH, provided at least one R¹¹ is COOH. In these embodiments, n can be 1-50, 1-40, 1-30, 1-20, 1-25, 1-20, 1-15, 1-10, or 1-5.

In embodiments, the linker includes a nitrogen-containing C₃₋₇heterocycle (e.g., pyrollidine, piperidine, piperazine), polyalkylene diamine (e.g., polyethylene diamine, polypropylene diamine, polybutylene diamine), a negatively charged amino acid (e.g., aspartic acid, glutamic acid, gamma-glutamic acid), or a saccharide (e.g., monosaccharide, polysaccharide, inositol, or polysialic acid).

In embodiments, the linker comprises polyalkylene oxide. The polyalkylene oxide can be linear or branched. For example the linker can include polyethylene glycol (e.g., PEG 40, PEG 100, PEG 150, PEG 200, PEG 300, PEG 2000), polypropylene glycol, or polybutylene glycol. The polyalkylene oxide can have a molecular weight of about 30 to about 5000 (e.g. about 30-4500, about 30-4000, about 30-3500, about 30-3000, about 30-2500, about 30-2000, about 30-1500, about 30-1000, about 46-1000, about 46-750, about 46-500, about 46-250, or about 46-100). Polyethylene oxide spacers, with and without reactive functionality at one or both of their termini, are well-known in the art and commercially available through, e.g., Quanta Biodesign, PierceNet, and SigmaAldrich.

Through the functional groups at the first and second termini, the linkers react with the fusion protein reactive enzymes site-specifically, in high yield, with rapid kinetics under mild conditions to provide at least a bivalent antibody construct. The functional groups at the linker termini can be any functional groups that irreversibly inhibit the reactive enzymes.

Examples of functional groups include, but are not limited to, p-nitrophenyl phosphonate, O⁶-benzylguanine, α-haloalkane or derivative thereof, haloaromatic compound or derivative thereof, beta-lactam or derivative thereof such as clavulanic acid or derivative thereof, aglycone or derivative thereof, hydroxamic acid-benzophenones or derivative thereof, cognate oriT oligonucleotide sequence, cysteine-reactive ATP-binding site inhibitors, quinone methides or derivative thereof, α-halo phosphonic acids or precursor or derivative thereof, formylchromones or derivative thereof, cognate RNA sequence, adenosine or derivative thereof, cytosine or derivative thereof, cognate DNA nicking sites, thiiranes or derivative thereof, hydroxamic acids or derivative thereof, α-ketoxazole inhibitor or derivative thereof, electrophilic steroid, phosphonates, carbamates, aromatic alkynes, beloranib or derivative thereof, and combinations of the foregoing.

In embodiments, the functional group is selected from the group consisting of p-nitrophenyl phosphonate, O⁶-benzylguanine, α-haloalkane or derivative thereof, haloaromatic compound or derivative thereof, beta-lactam or derivative thereof such as clavulanic acid or derivative thereof, aglycone or derivative thereof, hydroxamic acid-benzophenones or derivative thereof, cognate oriT oligonucleotide sequence, and combinations thereof. In embodiments, the functional group is selected from the group consisting of p-nitrophenyl phosphonate, O⁶-benzylguanine, α-haloalkane or derivative thereof, haloaromatic compound or derivative thereof, and combinations thereof.

In embodiments, the functional group is a cognate oriT oligonucleotide sequence. In refinements of the foregoing embodiment, the cognate oriT oligonucleotide sequence is selected from the group consisting of 5′-TTTGCGTAGTGTGTGGTGCTTT-3′ (SEQ ID NO: 1); 5′-TTTGCGTGGGGTGTGGTGCTTT-3′ (SEQ ID NO: 2); 5′-TTTGCGTAGGGTGTGGTGCTTT-3′ (SEQ ID NO: 3); 5′-CGCGCACCGAAAGGTGCGTATTGTCTATAGCCCAGATTTAAGGA-3′ (SEQ ID NO: 4); 5′-CCATTTCTCGAAGAGAAACCGGTAAATGCGCCCT-3′ (SEQ ID NO: 5); 5′-CACACACTTTATGAATATAAAGTATAGTGTTATACTTTA-3′ (SEQ ID NO: 6); 5′ACGTTTCTGAACGAAGTGAAGAAACGTCTAAGTGCGCCCT-3′ (SEQ ID NO: 7) and combinations thereof.

In embodiments, the fusion protein reactive enzyme is a relaxase domain of type I DNA topoisomerases and the functional group is a cognate oriT oligonucleotide sequence. In embodiments, the relaxase domain of type I DNA topoisomerases (identified by mobility plasmids for each relaxase protein sequence)/cognate oriT oligonucleotide sequence pair is selected from the group consisting of pR100/5′-TTTGCGTAGTGTGTGGTGCTTT-3′ (SEQ ID NO: 1); pF/5′-TTTGCGTGGGGTGTGGTGCTTT-3′ (SEQ ID NO: 2); TraI_{P307}/5′-TTTGCGTAGGGTGTGGTGCTTT-3′ (SEQ ID NO: 3); pR388/5′-CGCGCACCGAAAGGTGCGTATTGTCTATAGCCCAGATTTAAGGA-3′ (SEQ ID NO: 4); pR1162/5′-CCATTTCTCGAAGAGAAACCGGTAAATGCGCCCT-3′ (SEQ ID NO: 5); pMobMN199/5′-CACACACTTTATGAATATAAAGTATAGTGTTATACTTTA-3′ (SEQ ID NO: 6); pSC101/5′ACGTTTCTGAACGAAGTGAAGAAACGTCTAAGTGCGCCCT-3′ (SEQ ID NO: 7), and combinations thereof.

In embodiments, the first fusion protein reactive enzyme comprises cutinase and the functional group at the first terminus comprises a p-nitrophenyl phosphonate.

In embodiments, the first fusion protein reactive enzyme comprises SnapTag and the functional group at the first terminus comprises an O⁶-benzylguanine.

In embodiments, the first fusion protein reactive enzyme comprises HaloTag and the functional group at the first terminus comprises an α-chloroalkane.

In embodiments, the first fusion protein reactive enzyme comprises beta-lactamase and the functional group at the first terminus comprises a beta-lactam or derivative thereof.

In embodiments, the first fusion protein reactive enzyme comprises glycosidase and the functional group at the first terminus comprises an aglycone or a derivative thereof.

In embodiments, the first fusion protein reactive enzyme comprises matrix metalloproteinase and the functional group at the first terminus comprises hydroxamic acid-benzophenones or derivative thereof.

In embodiments, the first fusion protein reactive enzyme comprises a relaxase domain of type I DNA topoisomerases and the functional group at the first terminus comprises a cognate oriT oligonucleotide sequence.

In embodiments, the first fusion protein reactive enzyme comprises a cytoplasmic protein tyrosine kinase domain and the functional group at the first terminus comprises a cysteine-reactive ATP-binding site inhibitor.

In embodiments, the first fusion protein reactive enzyme comprises an alkaline phosphatase and the functional group at the first terminus comprises a functional group selected from the group consisting of quinone methides, α-halo phosphonic acid, precursor of the foregoing, derivative of the foregoing, and combinations of the foregoing.

In embodiments, the first fusion protein reactive enzyme comprises a protein-tyrosine-phosphatase and the functional group at the first terminus comprises a functional group selected from the group consisting of a formylchromone, α-halo phosphonic acid, precursor of the foregoing, derivative of the foregoing, and combinations of the foregoing.

In embodiments, the first fusion protein reactive enzyme comprises a mutant of 23S rRNA (adenine (2503)-C(2))-methyltransferase and the functional group at the first terminus comprises cognate RNA sequence.

In embodiments, the first fusion protein reactive enzyme comprises glucosidase and the functional group at the first terminus comprises aglycone or derivative thereof.

In embodiments, the first fusion protein reactive enzyme comprises N-6 adenine-specific DNA methylase and the functional group at the first terminus comprises adenosine or derivative thereof.

In embodiments, the first fusion protein reactive enzyme comprises N(4)-cytosine-specific DNA methylase and the functional group at the first terminus comprises cytosine or derivative thereof.

In embodiments, the first fusion protein reactive enzyme comprises DNA (cytosine-5-)-methyltransferase and the functional group at the first terminus comprises cytosine or derivative thereof.

In embodiments, the first fusion protein reactive enzyme comprises mutants of haloalkane dehalogenases and the functional group at the first terminus comprises a functional group selected from the group consisting of haloalkanes, haloaromatic compounds, or derivative thereof.

In embodiments, the first fusion protein reactive enzyme comprises HNH endonucleases and the functional group at the first terminus comprises cognate DNA nicking sites.

In embodiments, the first fusion protein reactive enzyme comprises nicking endonucleases and the functional group at the first terminus comprises cognate DNA nicking sites.

In embodiments, the first fusion protein reactive enzyme comprises gelatinase B and the functional group at the first terminus comprises a functional group selected from the group consisting of thiiranes, hydroxamic acids, derivatives of the foregoing, and combinations of the foregoing.

In embodiments, the first fusion protein reactive enzyme comprises gelatinase A and the functional group at the first terminus comprises a functional group selected from the group consisting of thiiranes, hydroxamic acids, derivatives of the foregoing, and combinations of the foregoing.

In embodiments, the first fusion protein reactive enzyme comprises stromelysins and the functional group at the first terminus comprises a functional group selected from the group consisting of thiiranes, hydroxamic acids, derivatives of the foregoing, and combinations of the foregoing.

In embodiments, the first fusion protein reactive enzyme comprises a fatty acid amide hydrolase and the functional group at the first terminus comprises an α-ketoxazole inhibitor or derivative thereof.

In embodiments, the first fusion protein reactive enzyme comprises esterases and the functional group at the first terminus comprises a functional group selected from the group consisting of phosphonates, carbamates, derivatives of the foregoing, and combinations of the foregoing.

In embodiments, the first fusion protein reactive enzyme comprises cytochrome P450s and the functional group at the first terminus comprises a functional group selected from the group consisting of electrophilic steroids, aromatic alkynes, derivatives of the foregoing, and combinations of the foregoing.

In embodiments, the first fusion protein reactive enzyme comprises methionine aminopeptidases and the functional group at the first terminus comprises beloranib or derivative thereof.

In embodiments, the second fusion protein reactive enzyme comprises cutinase and the functional group at the second terminus comprises a p-nitrophenyl phosphonate.

In embodiments, the second fusion protein reactive enzyme comprises SnapTag and the functional group at the second terminus comprises an O⁶-benzylguanine.

In embodiments, the second fusion protein reactive enzyme comprises HaloTag and the functional group at the second terminus comprises an α-chloroalkane.

In embodiments, the second fusion protein reactive enzyme comprises beta-lactamase and the functional group at the second terminus comprises a beta-lactam or a derivative thereof.

In embodiments, the second fusion protein reactive enzyme comprises glycosidase and the functional group at the second terminus comprises an aglycone or a derivative thereof.

In embodiments, the second fusion protein reactive enzyme comprises matrix metalloproteinase and the functional group at the second terminus comprises hydroxamic acid-benzophenone or a derivative thereof.

In embodiments, wherein the second fusion protein reactive enzyme comprises a relaxase domain of type I DNA topoisomerases and the functional group at the second terminus comprises a cognate oriT oligonucleotide sequence.

In embodiments, the second fusion protein reactive enzyme comprises a cytoplasmic protein tyrosine kinase domain and the functional group at the second terminus comprises a cysteine-reactive ATP-binding site inhibitor.

In embodiments, the second fusion protein reactive enzyme comprises an alkaline phosphatase and the functional group at the second terminus comprises a functional group selected from the group consisting of quinone methides, α-halo phosphonic acid, precursor of the foregoing, derivative of the foregoing, and combinations of the foregoing.

In embodiments, the second fusion protein reactive enzyme comprises a protein-tyrosine-phosphatase and the functional group at the second terminus comprises a functional group selected from the group consisting of a formylchromone, α-halo phosphonic acid, precursor of the foregoing, derivative of the foregoing, and combinations of the foregoing.

In embodiments, the second fusion protein reactive enzyme comprises a mutant of 23S rRNA (adenine (2503)-C(2))-methyltransferase and the functional group at the second terminus comprises cognate RNA sequence.

In embodiments, the second fusion protein reactive enzyme comprises glucosidase and the functional group at the second terminus comprises aglycone or derivative thereof.

In embodiments, the second fusion protein reactive enzyme comprises N-6 adenine-specific DNA methylase and the functional group at the second terminus comprises adenosine or derivative thereof.

In embodiments, the second fusion protein reactive enzyme comprises N(4)-cytosine-specific DNA methylase and the functional group at the second terminus comprises cytosine or derivative thereof.

In embodiments, the second fusion protein reactive enzyme comprises DNA (cytosine-5-)-methyltransferase and the functional group at the second terminus comprises cytosine or derivative thereof.

In embodiments, the second fusion protein reactive enzyme comprises mutants of haloalkane dehalogenases and the functional group at the second terminus comprises a functional group selected from the group consisting of haloalkanes, haloaromatic compounds, or derivative thereof.

In embodiments, the second fusion protein reactive enzyme comprises HNH endonucleases and the functional group at the second terminus comprises cognate DNA nicking sites.

In embodiments, the second fusion protein reactive enzyme comprises nicking endonucleases and the functional group at the second terminus comprises cognate DNA nicking sites.

In embodiments, the second fusion protein reactive enzyme comprises gelatinase B and the functional group at the second terminus comprises a functional group selected from the group consisting of thiiranes, hydroxamic acids, derivatives of the foregoing, and combinations of the foregoing.

In embodiments, the second fusion protein reactive enzyme comprises gelatinase A and the functional group at the second terminus comprises a functional group selected from the group consisting of thiiranes, hydroxamic acids, derivatives of the foregoing, and combinations of the foregoing.

In embodiments, the second fusion protein reactive enzyme comprises stromelysins and the functional group at the second terminus comprises a functional group selected from the group consisting of thiiranes, hydroxamic acids, derivatives of the foregoing, and combinations of the foregoing.

In embodiments, the second fusion protein reactive enzyme comprises a fatty acid amide hydrolase and the functional group at the second terminus comprises an α-ketoxazole inhibitor or derivative thereof.

In embodiments, the second fusion protein reactive enzyme comprises esterases and the functional group at the second terminus comprises a functional group selected from the group consisting of phosphonates, carbamates, derivatives of the foregoing, and combinations of the foregoing.

In embodiments, the second fusion protein reactive enzyme comprises cytochrome P450s and the functional group at the second terminus comprises a functional group selected from the group consisting of electrophilic steroids, aromatic alkynes, derivatives of the foregoing, and combinations of the foregoing.

In embodiments, the second fusion protein reactive enzyme comprises methionine aminopeptidases and the functional group at the second terminus comprises beloranib or derivative thereof.

### Methods of Preparing Constructs

Another aspect of the disclosure provides a method including (a) contacting a first fusion protein including an affinity reagent and a reactive enzyme with a linker including a functional group specific for irreversibly inhibiting the first fusion protein reactive enzyme at a first terminus thereby coupling the first fusion protein and the linker at the first terminus, and (b) contacting a second fusion protein including an affinity reagent and a reactive enzyme with a second terminus of the linker, the second terminus of the linker including a functional group specific for irreversibly inhibiting the second fusion protein reactive enzyme thereby coupling the second fusion protein and the linker at the second terminus.

As used herein and unless specified otherwise, “coupled,” “couple,” or “coupling” encompasses covalent bond formation, for example, through which the fusion protein can irreversibly associate with the functional group at the linker terminus.

The method disclosed here joins fusion proteins including affinity reagents and reactive enzymes in a modular fashion using small molecule linkers that have functional groups at the termini which react site specifically with the reactive enzymes in high yield with rapid kinetics under mild reaction conditions.

The methods disclosed herein provide one or more advantages, for example, providing a means by which an incredibly diverse population of therapeutic constructs can be generated in a modular fashion. Additionally, the preparation of antibody formats with expanded functionality is straightforward using the constructs of the disclosure, as no reactive amino acids need to be engineered/functionalized directly on an antibody scaffold. Further, the mild, rapid, and site-specific nature of the coupling of the fusion proteins and the linkers allows for diverse effector molecules to be attached to an antibody fragment in precise stoichiometry in high yield and away from the antibody-like fragment. Further still, molecules that cannot be expressed in homologous hosts with antibody fragments such as toxins or glycosylated proteins can be expressed in various organisms and attached in a modular fashion to the construct. Modular synthesis permits the creation of molecules too large or complex to prepare using standard protein engineering/expression as such methods require polypeptides to be expressed as one or more chains in culture.

The chemical linking modular approach enables an efficient method to produce precisely-defined protein constructs of variable stoichiometry, orientation, and geometry. These attributes can be systematically altered by encoding diversity into the linkers and/or by altering the order of attachment of the fusion proteins to the linkers. Current protein engineering methods are limited to the intrinsic repertoire of natural polypeptide folds/peptide linkers to achieve a desired construct. Therefore, non-natural formats that may exhibit greater efficacy cannot be prepared using conventional engineering methods.

Finally, the expression of smaller antibody-like fragments enables expression in prokaryotic hosts in addition to the eukaryotic hosts typically used in the industrial production of antibody molecules. It is well known that the robustness of prokaryotes, in addition to product yields that are often 5× those of eukaryotic expression systems, enables a lower cost manufacturing platform.

Another aspect of the disclosure provides a construct prepared by the method of the disclosure.

In embodiments, step (a) and (b) are performed sequentially (i.e., stepwise). In embodiments, step (a) and (b) are performed contemporaneously (i.e., one pot). Each affinity reagent and reactive enzyme can be selected from any of the affinity reagents and reactive enzymes disclosed herein. The linker, including the functional group at the first terminus and the functional group at the second terminus, can be selected from any of the linkers and functional groups disclosed herein.

In embodiments, the first fusion protein reactive enzyme and the second fusion protein reactive enzyme are the same and the functional group specific for irreversibly inhibiting the first fusion protein reactive enzyme at the first terminus of the linker is the same as the functional group specific for irreversibly inhibiting the second fusion protein reactive enzyme at the second terminus of the linker, and steps (a) and (b) are performed contemporaneously.

In embodiments, the first fusion protein reactive enzyme and the second fusion protein reactive enzyme are the same and the functional group specific for irreversibly inhibiting the first fusion protein reactive enzyme at the first terminus of the linker is different from the functional group specific for irreversibly inhibiting the second fusion protein reactive enzyme at the second terminus of the linker, and steps (a) and (b) are performed contemporaneously.

In embodiments, the first fusion protein reactive enzyme and the second fusion protein reactive enzyme are different and the functional group specific for irreversibly inhibiting the first fusion protein reactive enzyme at the first terminus of the linker is the same as the functional group specific for irreversibly inhibiting the second fusion protein reactive enzyme at the second terminus of the linker, and steps (a) and (b) are performed sequentially.

In embodiments, the first fusion protein reactive enzyme and the second fusion protein reactive enzyme are different and the functional group specific for irreversibly inhibiting the first fusion protein reactive enzyme at the first terminus of the linker is the same as the functional group specific for irreversibly inhibiting the second fusion protein reactive enzyme at the second terminus of the linker, and steps (a) and (b) are performed contemporaneously.

In embodiments, the first fusion protein reactive enzyme and the second fusion protein reactive enzyme are different and the functional group specific for irreversibly inhibiting the first fusion protein reactive enzyme at the first terminus of the linker is different from the functional group specific for irreversibly inhibiting the second fusion protein reactive enzyme at the second terminus of the linker, and steps (a) and (b) are performed contemporaneously.

In embodiments, the first fusion protein reactive enzyme and the second fusion protein reactive enzyme are different and the functional group specific for irreversibly inhibiting the first fusion protein reactive enzyme at the first terminus of the linker is different from the functional group specific for irreversibly inhibiting the second fusion protein reactive enzyme at the second terminus of the linker, and steps (a) and (b) are performed sequentially.

The method disclosed herein provides one or more advantages such as allowing the scalable preparation of molecules with molecular weights greater than about 100 kDa, about 150 kDa, about 200 kDa, about 250 kDa and/or about 300 kDa and/or creation of diverse libraries of antibody-like drugs with altered valency, geometry, effector function, and stoichiometry simply by the combination of a comparatively smaller number of building blocks.

Steps (a) and (b) can be performed in any aqueous solution buffered to a physiological pH, for example, phosphate-buffered saline (PBS) at pH 7.4. The concentration of the first fusion protein, second fusion protein, and linker can generally be any concentration. The concentration is typically chosen such that the first fusion protein, second fusion protein, and linker are fully soluble in a chosen solvent, without forming saturated solutions. When the linker comprises only a functional group specific for irreversibly inhibiting an enzyme at a first terminus and a second terminus, the relative concentration of first fusion protein to linker to second fusion protein is at least about 1:1:1 to provide one functional group specific for irreversibly inhibiting an enzyme linker terminus per fusion protein.

In embodiments, steps (a) and (b) are performed at ambient temperatures. The temperature should not be so high as to denature any proteins present in the fusion proteins.

In embodiments, the constructs can be prepared on a solid support. In embodiments, one or more components of step (a) and/or (b) can be provided on a solid support. Suitable solid supports are well known in the art.

### Methods of Using the Constructs

Another aspect of the disclosure provides a method comprising administering a construct of the disclosure to a patient in need thereof. Yet another aspect of the disclosure provide uses of the construct of the disclosure as a medicament for a patient in need thereof. Another aspect of the disclosure provides the use of the construct of the disclosure as a diagnostic.

In embodiments, the patient suffers from a disorder or disease selected from the group consisting of breast cancer, inhalational anthrax, rheumatoid arthritis, systemic juvenile idiopathic arthritis, Hodgkin lymphoma, chronic lymphocytic leukemia, follicular non-Hodgkin's lymphoma, diffuse large B cell lymphoma, relapsing remitting multiple sclerosis, systemic lupus erythematosus, gastric or gastro-esophageal junction adenocarcinoma, metastatic non-small-cell lung carcinoma, ulcerative colitis, Crohn's disease, melanoma, macular degeneration, osteoporosis, treatment-induced bone loss, metastases to bone, giant cell tumor of bone, malignant ascites, psoriatic arthritis, ankylosing spondylitis, metastatic renal cell cancer, prostate cancer, ovarian cancer, multiple myeloma, Castleman's disease, colorectal cancer, and combinations of the foregoing.

As used herein and unless specified otherwise, “disease” and “disorder” are used interchangeably.

In embodiments, at least one antibody or fragment thereof of the construct includes trastuzumab or fragment thereof, raxibacamab or fragment thereof, tocilizumab or fragment thereof, brentuximab or fragment thereof, ofatumumab or fragment thereof, belimumab or fragment thereof, ramucirumab or fragment thereof, vedolizumab or fragment thereof, obinutuzumab or fragment thereof, pembrolizumab or fragment thereof, ranibizumab or fragment thereof, pertuzumab or fragment thereof, denosumab or fragment thereof, catumaxomab or fragment thereof, golimumab or fragment thereof, siltuximab or fragment thereof, natalizumab or fragment thereof, panitumumab or fragment thereof, denosumab or fragment thereof, and combinations of the foregoing.

In embodiments, the patient suffers from breast cancer and at least one antibody or fragment thereof of the construct comprises trastuzumab or a fragment thereof.

In embodiments, the patient suffers from inhalational anthrax and at least one antibody or fragment thereof of the construct comprises raxibacumab or fragment thereof.

In embodiments, the patient suffers from rheumatoid arthritis or systemic juvenile idiopathic arthritis and at least one antibody or fragment thereof of the construct comprises tocilizumab or fragment thereof.

In embodiments, the patient suffers from Hodgkin lymphoma or systemic anaplastic large cell lymphoma and at least one antibody or fragment thereof of the construct comprises brentuximab or fragment thereof.

In embodiments, the patient suffers from a condition selected from the group consisting of chronic lymphocytic leukemia, follicular non-Hodgkin's lymphoma, diffuse large B cell lymphoma, rheumatoid arthritis, and relapsing remitting multiple sclerosis, and at least one antibody or fragment thereof of the construct comprises ofatumumab or fragment thereof.

In embodiments, the patient suffers from systemic lupus erythematosus and at least one antibody or fragment thereof of the construct comprises belimumab or fragment thereof.

In embodiments, the patient suffers from gastric or gastro-esophageal junction adenocarcinoma or metastatic non-small-cell lung carcinoma and at least one antibody or fragment thereof of the construct comprises ramucirumab or fragment thereof.

In embodiments, the patient suffers from ulcerative colitis or Crohn's disease and at least one antibody or fragment thereof of the construct comprises vedolizumab or fragment thereof.

In embodiments, the patient suffers from chronic lymphocytic leukemia or follicular lymphoma and at least one antibody or fragment thereof of the construct comprises obinutuzumab or fragment thereof.

In embodiments, the patient suffers from melanoma or metastatic non-small cell lung cancer and at least one antibody or fragment thereof of the construct comprises pembrolizumab or fragment thereof.

In embodiments, the patient suffers from macular degeneration and at least one antibody or fragment thereof of the construct comprises ranibizumab or fragment thereof.

In embodiments, the patient suffers from breast cancer and at least one antibody or fragment thereof of the construct comprises pertuzumab or fragment thereof.

In embodiments, the patient suffers from a condition selected from the group consisting of osteoporosis, treatment-induced bone loss, metastases to bone, and giant cell tumor of bone and at least one antibody or fragment thereof of the construct comprises denosumab or fragment thereof.

In embodiments, the patient suffers from malignant ascites and at least one antibody or fragment thereof of the construct comprises catumaxomab or fragment thereof.

In embodiments, the patient suffers from a condition selected from the group consisting of rheumatoid arthritis, psoriatic arthritis, ankylosing spondylitis, and ulcerative colitis and at least one antibody or fragment thereof of the construct comprises golimumab or fragment thereof.

In embodiments, the patient suffers from a condition selected from the group consisting of metastatic renal cell cancer, prostate cancer, ovarian cancer, non-Hodgkin's lymphoma, multiple myeloma, and Castleman's disease and at least one antibody or fragment thereof of the construct comprises siltuximab or fragment thereof.

In embodiments, the patient suffers from multiple sclerosis or Crohn's disease and at least one antibody or fragment thereof of the construct comprises natalizumab or fragment thereof.

In embodiments, the patient suffers from colorectal cancer and at least one antibody or fragment thereof of the construct comprises panitumumab or fragment thereof.

In embodiments, the patient suffers from a condition selected from the group consisting of osteoporosis, treatment-induced bone loss, metastases to bone, and giant cell tumor of bone and at least one antibody or fragment thereof of the construct comprises denosumab or fragment thereof.

The constructs, methods, and uses in accordance with the disclosure can be better understood in light of the following examples, which are merely intended to illustrate the constructs and are not meant to limit the scope thereof in any way.

### EXAMPLES

### Example 1

Gene/Protein Expression Vector Construction of F(ab) Domains from Trastuzumab

Genes encoding the F(ab) domains (light chain variable V_{L}—light chain constant kappa chain C_{K}, and heavy chain variable domain V_{H}—heavy chain constant domain C_{H}1) from monocolonal antibody trastuzumab (hereafter designated “TFab(s)”) were synthesized with codon optimization for E. coli by Genscript. The following eight dicistronic fusion proteins were assembled in a tetracycline-inducible expression vector based on pASK-IBA32 (IBA Biosciences) using restriction free PCR:

1.
(SEQ ID NOs: 8 & 9)
“N-terminal cutinase-V_{L} fusion”-5′-RBS1-ATG-cutinase-(EAAAK)₂-
V_{L}C_{K}-**-RBS2-ATG-V_{H}C_{H}1-LVPRGS-HHHHHH-**-3′
2.
(SEQ ID NOs: 10 & 11)
“N-terminal SnapTag-V_{L} fusion”-5′-RBS1-ATG-SnapTag-(EAAAK)₂-
VLCK-RBS2-ATG-V_{H}C_{H}1-LVPRGS-HHHHHH-**-3′
3.
(SEQ ID NOs: 12 & 13)
“C-terminal cutinase-C_{K} fusion”-5′-RBS1-ATG-V_{L}C_{K}-(EAAAK)₂-cutinase-
**-RBS2-ATG-V_{H}C_{H}1-LVPRGS-HHHHHH-**-3′
4.
(SEQ ID NO: 14 & 15)
“C-terminal SnapTag-C_{K} fusion”-5′-RBS1-ATG-V_{L}C_{K}-(EAAAK)₂-SnapTag-**-
RBS2-ATG-V_{H}C_{H}1-LVPRGS-HHHHHH-**-3′
5.
(SEQ ID NO: 16 & 17)
“N-terminal cutinase-V_{H} fusion”-5′-RBS1-ATG-V_{L}C_{K}-**-RBS2-ATG-
cutinase-(EAAAK)₂-V_{H}C_{H}1-LVPRGS-HHHHHH-**-3′
6.
(SEQ ID NO: 18 & 19)
“N-terminal SnapTag-V_{H} fusion”-5′-RBS1-ATG-V_{L}C_{K}-**-RBS2-ATG-SnapTag-
(EAAAK)₂-V_{H}C_{H}1-LVPRGS-HHHHHH-**-3′
7.
(SEQ ID NO: 20 & 21)
“C-terminal cutinase-C_{H}1 fusion”-5′-RBS1-ATG-V_{L}C_{K}-**-RBS2-ATG-V_{H}C_{H}1-
(EAAAK)₂-cutinase-LVPRGS-HHHHHH-**-3′
8.
(SEQ ID NO: 22 & 23)
“C-terminal SnapTag-C_{H}1 fusion”-5′-RBS1-ATG-V_{L}C_{K}-**-RBS2-ATG-V_{H}C_{H}1-
(EAAAK)₂-SnapTag-LVPRGS-HHHHHH-**-3′

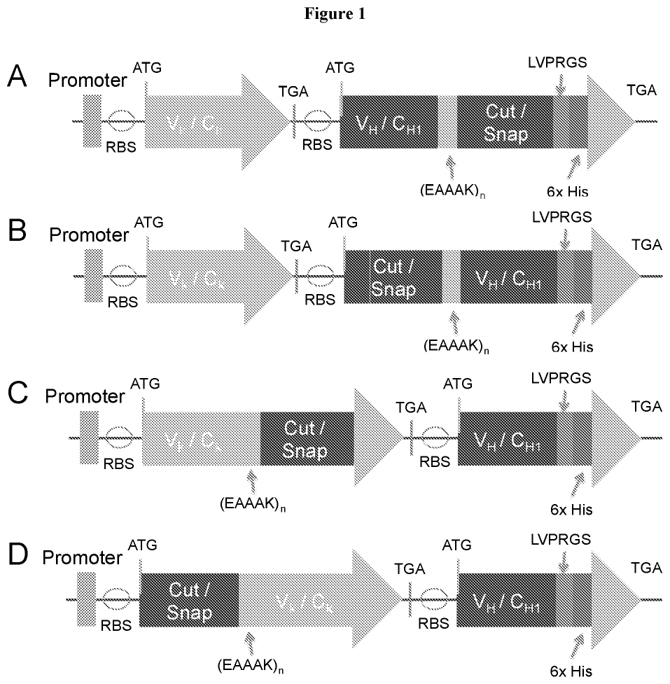

FIG. 1 shows the Trastuzumab F(ab) (TFab) fusion gene/protein constructs. A) C-terminal heavy chain fusions. B) N-terminal heavy chain fusions. C) C-terminal light chain fusions. D) N-terminal light chain fusions.

### Example 2

### Protein Expression/Purification

Vectors encoding the eight TFab fusion proteins of Example 1 were transformed into Shuffle E. coli (New England Biolabs). Expression cultures containing growth media (500 mL 2xYT), carbenicillin (200 μg/mL) and spectinomycin (50 μg/mL) were inoculated with an overnight culture (5 mL) of cell stocks bearing the appropriate expression cassette. Cultures were grown at 30° C. with shaking at 250 rpm until reaching an optical dispersion (OD) of about 0.8. At this point, anhydrotetracycline (aTc) was added to the cultures at final concentration of 200 ng/mL to induce protein expression. Cultures were maintained at 24° C. with shaking at 250 rpm for 14 hr during the expression phase.

After expression, cells were harvested via centrifugation and lysed via chemical disruption using 35 mL CelLytic B (Sigma Aldrich) containing DNAse I, and EDTA-free protease inhibitors (Roche). Cell lysates were cleared of debris by centrifugation and added to a column containing cobalt IMAC resin (3 mL bed volume). Lysates were incubated for an hour over the beads with agitation at 4° C. After this period, beads were washed extensively with phosphate buffered saline (PBS) and the products then eluted with imidazole containing buffer (20 mL). Protein eluates were then purified first on a protein L column (GE Healthcare) followed by size exclusion chromatography (SEC) on a Superdex 200 column (GE Healthcare) via a fast protein liquid chromatography (FPLC) instrument. All column purifications via FPLC were done according to manufacturer's suggested protocols. Fractions containing pure protein products were pooled and concentrated in centrifugal concentrators prior to linking reactions.

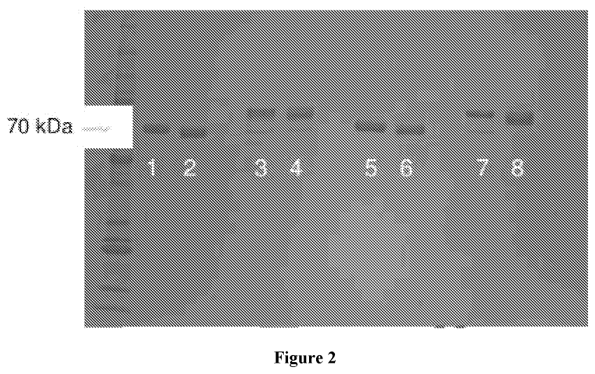

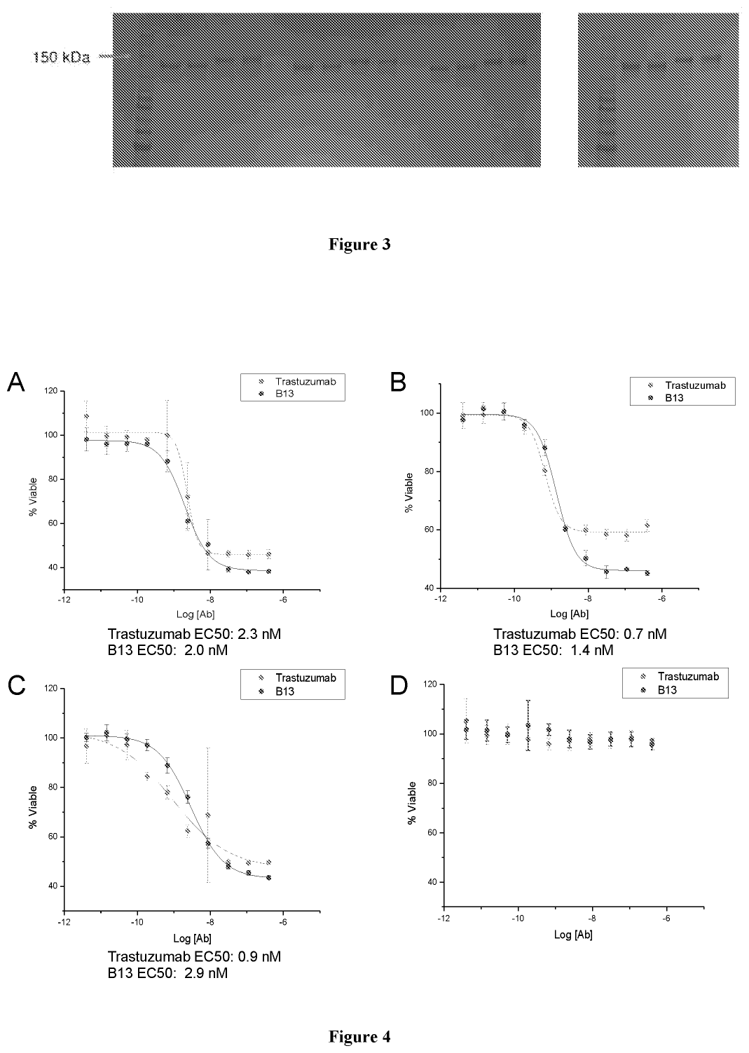

FIG. 2 shows the sodium dodecylsuflate polyacrylamide (SDS-PAGE) gel of purified TFab-cutinase and TFab-SnapTag fusions. Lanes 1 & 2 show bands near the calculated molecular weights for the N-terminal V_{H} fusions of cutinase (72 kDa) and SnapTag (69 kDa) respectively. Lanes 3 & 4 show bands from C-terminal V_{H} fusions. Lanes 5 & 6 and 7 & 8, show bands from C-terminal C_{K} and C_{H1} fusions, respectively.

Thus, Examples 1 and 2 show the preparation of fusion proteins of the disclosure comprising an affinity reagent (TFab) and a reactive enzyme (cutinase and/or SnapTag).

### Example 3

Preparation of a Linker for Joining a Cutinase-TFab Fusion Protein with a SnapTag-TFab Fusion Protein

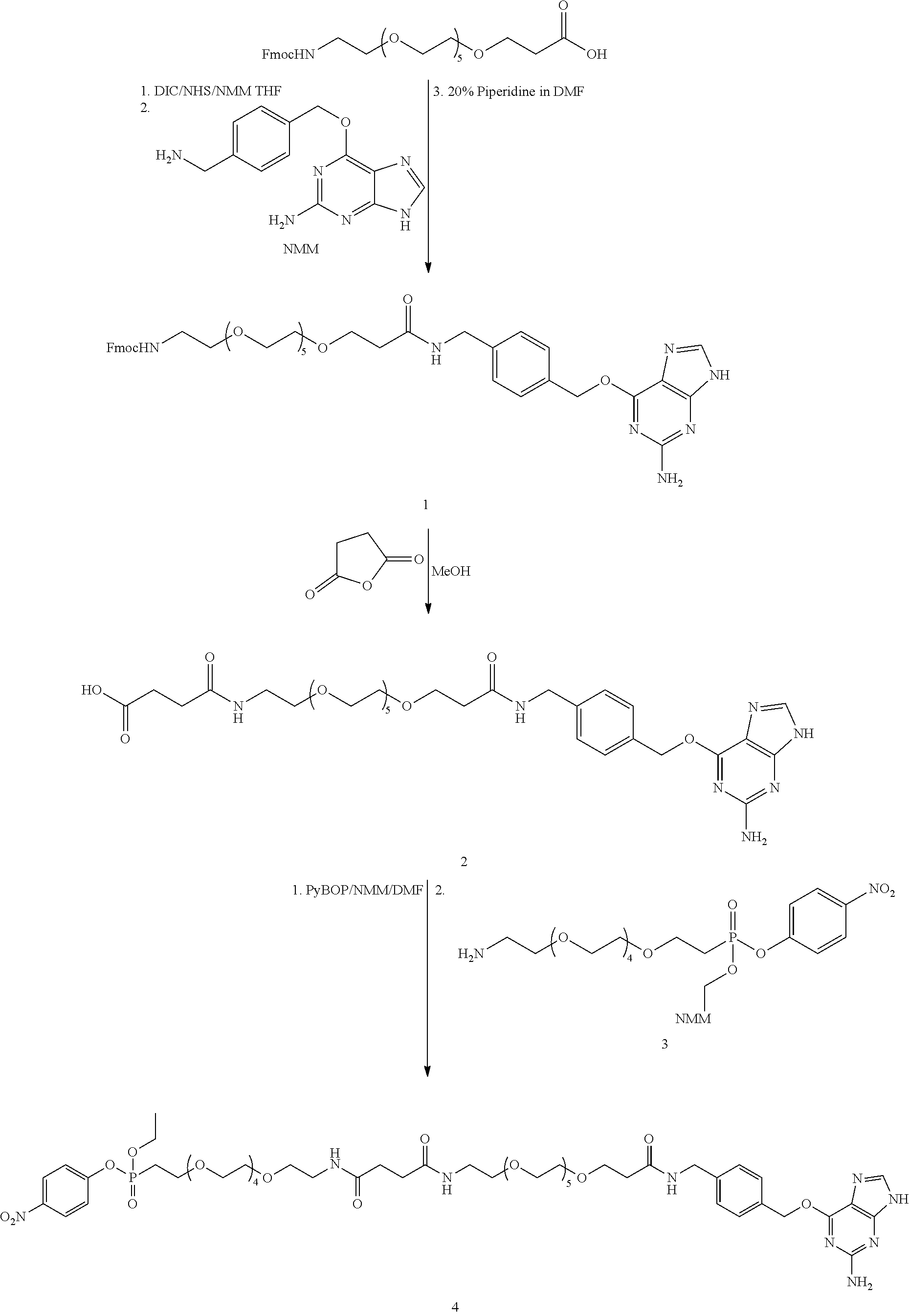

A linker according to the disclosure comprising a p-nitrophenyl phosphonate at the first terminus, an O⁶-benzylguanine at the second terminus, and ethylene glycol and amide units was prepared according to the synthetic scheme shown below.

Preparation of 1-amino-N-(4-(((2-amino-9H-purin-6-yl)oxy)methyl)benzyl)-3, 6,9,12,15,18-hexaoxahenicosan-21-amide (1). Fmoc-21-amino-4,7, 10, 13, 16, 19-hexaoxaheneicosanoic acid (567 mg, 0.99 mmol) was dissolved in 5 mL THF. To this solution was added DIC (149 mg, 183 μL, 1.18 mmol) followed by NHS (136 mg, 1.18 mmol) and the reaction was allowed to stir overnight at room temperature. The solvent was removed on a rotary evaporator and the resulting yellow oil was resuspended in 5 mL DMF. To this solution was added (4-aminomethyl)-O⁶-benzylguanine (266 mg, 0.99 mmol) and the reaction allowed to stir 2 days at room temperature. After this period, the DMF was stripped from the reaction under a stream of N₂ overnight. The resulting orange oil was then treated with 5 mL 20% piperidine in MeOH and allowed to stir 1 hr at room temperature. The solvent was removed using a rotary evaporator and the resulting orange oil resuspended in 10 mL H₂O. This solution was then centrifuged to pellet insoluble material and the clarified supernantant applied in 5 separate portions to a reversed phase C₁₈ column via a Waters Delta 400 HPLC. Elution was carried out using a linear gradient of 75% ACN in deionized ultra-filtered water (DIUF)+0.01% TFA over 60 min with DIUF+0.01% TFA as the mobile phase at a flow rate of 10 mL/min. Fractions from 20-35 min were then analyzed by MALDI-MS. Pure fractions were pooled and lyophilized to yield an amber oil. (424 mg, 71%)

Preparation of 1-(4-4-(((2-amino-9H-purin-6-yl)oxy)methyl)phenyl)-3, 25-dioxo-6,9,12,15,18,21-hexaoxa-2,24-diazaoctacosan-28-oic acid (2). Compound 1 (50 mg, 83 μmol) was dissolved in 2.5 mL MeOH and to this solution was added succinic anhydride (8.3 mg, 83 μmol). Upon completion of the reaction (˜30 min as monitored by MALDI-MS), the solvent was removed on a rotary evaporator. The resulting amber oil was then purified via silica gel chromatography first using 10:1 CH₂Cl₂: MeOH to remove impurities and then by elution with MeOH to yield a gummy amber solid. (42 mg, 72%)

The preparation of intermediate (3), 17-(ethoxy(4-nitrophenoxy)phosphoryl)-3, 6,9,12,15-pentaoxyaheptadecan-1-aminium chloride, is described in Modica, J.A. et al., Chembiochem. 2012 Nov. 5; 13(16):2331-4, which is hereby incorporated by reference in its entirety.

Preparation of ethyl (4-nitrophenyl) (1-(4- (((2-amino-9H-purin-6-yl) oxy)methyl)phenyl)-3,25,28-trioxo-6,9,12,15,18,21,32,35,38,41,44-undecaoxa-2,24,29-triazahexatetracontan-46-yl)phosphonate (4). Compound 2 (39 mg, 55 μmol) was dissolved in 1 mL DMF. To this solution was added PyBOP (34 mg, 58 μmol) followed by N-methylmorpholine (5.6 mg, 6.1 μL, 55 μmol) and the reaction was allowed to stir for 30 min at room temperature. After this period, compound 3 (29.3 mg, 55 μmol) dissolved in 250 μL DMF was added to the mixture followed by N-methylmorpholine (11.2 mg, 12.2 μL, 110 μmol) and the reaction was allowed to stir overnight (˜16 hr). After this period, the mixture was diluted to 5 mL using DIUF and the mixture centrifuged to remove insoluble material. The clarified supernatant was then and injected onto a C₁₈ semi-prep scale column via a Waters Delta 4000 HPLC and purified using a linear gradient of 75% aqueous ACN+0.1% TFA over 70 min with DIUF+0.1% TFA as the mobile phase at a flow rate of 10 mL/min. Fractions were collected at a rate of 1/min. Fractions from 25-40 min were then analyzed by MALDI. Those containing pure product were pooled, frozen at −80° C. and lyophilized to yield a yellow oil. This oil was then subjected to one additional round of HPLC purification using the aforementioned procedure. Pure fractions from this run were pooled, frozen and lyophilized to yield a light yellow oil (11 mg, 17%).

Thus, Example 3 shows the preparation of a linker of the disclosure comprising a first functional group specific for irreversibly inhibiting a first fusion protein reactive enzyme at a first terminus, and a second functional group specific for irreversibly inhibiting a second fusion protein reactive enzyme at a second terminus.

### Example 4

### Di-TFab Construct Assembly

Equal volumes of cutinase-TFab fusion (5 μM) and SnapTag-TFab fusion (5 μM) from Example 2 and cutinase-SnapTag linker (5 μM) from Example 3 in PBS pH 7.4 were mixed and allowed to react for 4 hr. After the reaction, products were purified via size-exclusion chromatography.

FIG. 5 shows SDS-PAGE gels of the constructs prepared. From a pool of 8 TFabs and one linker, 16 bivalent analog constructs can be produced.

Thus, Example 4 shows preparation of a library of constructs of the disclosure comprising a first fusion protein, a second fusion protein, and a linker, wherein the first fusion protein and the second fusion protein each comprises an affinity reagent and a reactive enzyme; and the linker comprises a first functional group specific for irreversibly inhibiting the first fusion protein reactive enzyme at a first terminus, and a second functional group specific for irreversibly inhibiting the second fusion protein reactive enzyme at a second terminus.

### Example 5

### Cell Growth Inhibition Assay

Trastuzumab is employed in the treatment of HER2+ breast cancers. Two HER2 (+++) cell lines (BT474, SKBR3), one HER2 (++) cell line (MDA-MB-VII-135) and one control cell line AT-431 (HER2−) were cultured according to established protocols and treated with doses of Trastuzumab or one of the bivalent trastuzumab analogs prepared in Example 4 in concentrations ranging from 4 pM to 4 μM for 96 h. Viability after this period was determined using the Alamar blue assay (Life Technologies). Viabilities were calculated as percentage values vs. untreated controls.

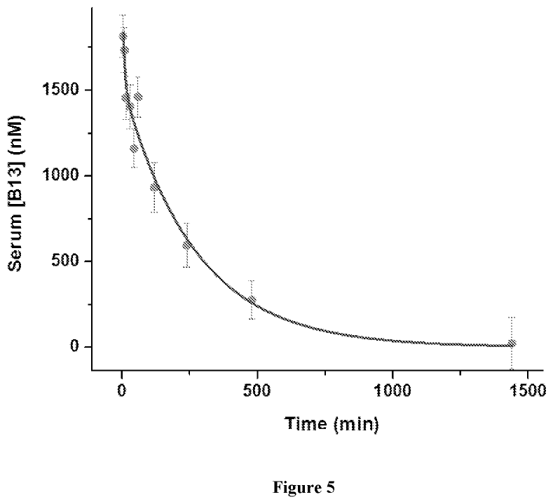

From these experiments, one construct, designated B-13, showed the most favorable cell growth inhibition and EC50 value vs. Trastuzumab. Cell viability experiments were performed again with B-13 vs. Trastuzumab. FIG. 4 shows the cell viability assay data showing comparable cell growth inhibition by trastuzumab and B-13 in various cell lines. A) Growth inhibition of BT474 cells. B) Growth inhibition of SKBR3 cells. C) Growth inhibition of MDA-MB-135-VII cells. D) Control experiment using HER2(−) AT-431cells showing no growth inhibition for trastuzumab or B-13.

Thus, Example 5 shows how libraries of constructs of the disclosure can be assayed for biological uses, for example, cell growth inhibition. Further, Example 5 shows cytotoxicity of the constructs in in vitro cancer cell viability assays. The fusion protein construct assembly method of Example 3 enables the preparation of multifunctional antibody-like therapeutic molecules in a one pot fashion.

### Example 6

### In Vivo Pharmacokinetics of Di-TFab Constructs

The B-13 construct (2 mg/mL) was administered as an IV bolus to SCID-beige mice at a total dose of 10 mg/kg. Blood samples (˜200 μL) were drawn from tail veins at various time points post administration. A total of three mice (n=3) were treated and sampled for each time point. An ELISA method was used to determine the concentration of B13 in the serum via standard curve comparison. As shown in FIG. 5, these data were plotted and fitted to a two compartment (biexponential) pharmacokinetic model to yield an elimination rate constant of 0.22 hr⁻¹ and a half-life of 3.1 hr.

Thus, Example 6 shows in vivo use of the fusion protein constructs of the disclosure and the pharmacokinetic data of a construct of the disclosure.

### Example 7

### Effect of B-13 Administration on Mice Bearing BT474 Tumor Xenografts

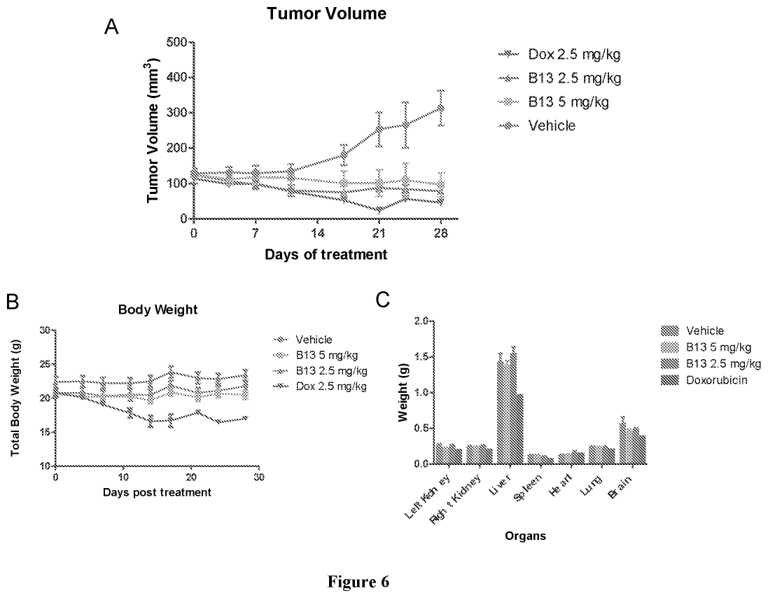

Eight-week-old female SCID beige mice were obtained from Charles River Laboratories and implanted with 0.025-mg, 90-day release, 17β-estradiol pellets (Innovative Research of America). After 2 days, 2×10⁶ BT474 cells were resuspended in 100 μl of PBS and 1:1 mixture with Matrigel was inoculated orthotopically into the mammary gland fat pad. Once tumors reached a volume of ˜150 mm³, 20 animals were randomly assigned into four treatment cohorts (n=5) with equal average tumor volumes. In one cohort, B13 was administered by intraperitoneal (IP) injection five times per week (Monday-Friday) over 4 weeks in a total of twenty doses of 5 mg kg 1; in a second cohort, B13 was administered via the same route and frequency at a dose of 2.5 mg/kg. In a third cohort, doxorubicin (dox), was administered via IP injection twice weekly in a total of eight doses over 4 weeks at 2.5 mg/kg. A control cohort was injected five times per week with vehicle (PBS+.004% (w/w) Polysorbate 20). B13 and dox solutions were both formulated in the same vehicle. B13 samples contained<1.0 endotoxin units (EU)/mL as determined by limulus amebocyte lysate (LAL) assay. Tumor dimensions were serially measured every 2 days, and volumes calculated using the formula V=(L×W²)/2, where V′=volume, L=length and W=width. The results are shown in FIG. 6.

B13 shows significant anti-tumor activity in a BT474 mouse xenograft model. Furthermore, B13, at the two doses used in this study, shows no outward signs of toxicity. This is in contrast to dox where the treatment cohort showed significant weight loss.

The foregoing description is given for clearness of understanding only, and no unnecessary limitations should be understood therefrom, as modifications within the scope of the invention may be apparent to those having ordinary skill in the art.

1-171. (canceled)

172. A construct comprising
a first fusion protein, a second fusion protein, and a linker,
wherein
the first fusion protein comprises a first reactive enzyme and the second fusion protein comprises a second reactive enzyme; and
the linker comprises a first functional group specific for irreversibly inhibiting the first reactive enzyme at a first terminus, and a second functional group specific for irreversibly inhibiting the second reactive enzyme at a second terminus, wherein the first functional group specific for irreversibly inhibiting the first reactive enzyme is coupled to the first reactive enzyme and the second functional group specific for irreversibly inhibiting the second reactive enzyme is coupled to the second reactive enzyme, such that the first fusion protein and second fusion protein are linked in the form of the construct, and
wherein the first reactive enzyme comprises cutinase and the second reactive enzyme comprises HaloTag or SnapTag.

173. The construct of claim 172, wherein the first fusion protein further comprises a first affinity reagent and the second fusion protein further comprises a second affinity reagent.

174. The construct of claim 173, wherein the first fusion protein affinity reagent and second fusion protein affinity reagent are different.

175. The construct of claim 173, wherein the first and second affinity reagents are independently selected from the group consisting of antibody or fragment thereof, small molecule, monobody, protein, and combinations thereof.

176. The construct of claim 175, wherein the first affinity reagent comprises an antibody or fragment thereof and the antibody or fragment thereof is selected from the group consisting of a light chain variable domain (V_{L}), a light chain constant domain (C_{L}), a heavy chain variable domain (V_{H}), a heavy chain constant domain (C_{H}1), and a combination thereof.

177. The construct of claim 175, wherein the first affinity reagent comprises an antibody or fragment thereof and the antibody or fragment thereof is selected from the group consisting of adalimumab, alemtuzumab, arcitumomab, cetuximab, trastuzumab, imciromab, capromab, infliximab, abciximab, rituximab, basiliximab, palivizumab, nofetumomab, omalizumab, daclizumab, ibritumomab tiuxetan, muromonab, edrecolomab gemtuzumab ozogamicin, golimumab, certolizumab, eculizumab, ustekinumab, panitumumab, tositumomab, bevacizumab, raxibacumab, tocilizumab, brentuximab, ofatumumab, belimumab, ramucirumab, vedolizumab, obinutuzumab, pembrolizumab, ranibizumab, pertuzumab, denosumab, catumaxomab, golimumab, siltuximab, natalizumab, panitumumab, and denosumab.

178. The construct of claim 175, wherein the small molecule is a drug.

179. The construct of claim 178, wherein the small molecule is selected from the group consisting of aldesleukin, alendronic acid, alfaferone, alitretinoin, allopurinol, aloprim, aloxi, altretamine, aminoglutethimide, L-asparaginase, amifostine, amrubicin, amsacrine, anastrozole, anzmet, aranesp, arglabin, arsenic trioxide, aromasin, 5-azacytidine, azathioprine, BCG or tice-BCG, bestatin, betamethasone acetate, betamethasone sodium phosphate, bexarotene, bleomycin sulphate, broxuridine, bortezomib, bleomycin, busulfan, calcitonin, campath, capecitabine, carboplatin, carmustine, casodex, cefesone, celmoleukin, cerubidin, chlorambucil, cisplatin, colaspase, cladribin, clodronic acid, cyclophosphamide, cytarabine, dacarbazine, dactinomycin, daunoxome, decadron, decadron phosphate, delestrogen, denileukin diftitox, depomedrol, deslorelin, dexrazoxane, daunorubicin, diethylstilbestrol, 2′,2′-difluorodeoxycytidine, diflucan, docetaxel, doxifluridine, doxorubicin, dronabinol, DW-166HC, eligard, elitek, ellence, emend, epirubicin, epoetin-alfa, epogen, eptaplatin, ergamisol, estrace, estradiol, estramustine sodium phosphate, ethinylestradiol, ethyol, etidronic acid, etopophos, etoposide, fadrozole, farstone, filgrastim, finasteride, fligrastim, floxuridine, fluconazole, fludarabin, fludarabin phosphate, 5-fluorodeoxyuridine, 5-fluorodeoxyuridine monophosphate, 5-fluorouracil (5-FU), fluoxymesterone, flutamide, hexamethylmelamine, formestane, fosteabine, fotemustine, fulvestrant, gammagard, gemcitabine, gemtuzumab, gleevec, gliadel, goserelin, granisetron hydrochloride, histrelin, hycamtin, hydrocortone, erythro-hydroxynonyladenine, hydroxyurea, hydroxyprogesterone caproate, ibritumomab tiuxetan, idarubicin, ifosfamide, interferon-alpha, interferon-alpha-2, interferon-alpha-2α, interferon-alpha-2β, interferon-alpha-n1, interferon-alpha-n3, interferon-beta, interferon-gamma-1α, interleukin-2, intron A, iressa, irinotecan, kytril, lentinan sulphate, letrozole, leucovorin, leuprolide, leuprolide acetate, levamisole, levofolic acid calcium salt, levothroid, levoxyl, lomustine, lonidamine, marinol, mechlorethamine, mecobalamin, medroxyprogesterone acetate, megestrol acetate, melphalan, menest, 6-mercaptopurine, mesna, methotrexate, metvix, miltefosine, minocycline, mitomycin C, mitotane, mitoxantrone, modrenal, myocet, nedaplatin, neulasta, neumega, neupogen, nilutamide, nolvadex, NSC-631570, OCT-43, octreotide, ondansetron hydrochloride, orapred, oxaliplatin, paclitaxel, pediapred, pegaspargase, pegasys, pentostatin, N-phosphonoacetyl L-aspartate (PALA), picibanil, pilocarpine hydrochloride, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofiran, sobuzoxane, solu-medrol, streptozocin, strontium-89 chloride, Synthroid, tamoxifen, tamsulosin, tasonermin, tastolactone, taxoter, teceleukin, temozolomide, teniposide, testosterone propionate, testred, thioguanine, thiotepa, thyrotropin, tiludronic acid, topotecan, toremifen, tositumomab, tastuzumab, teosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin-stimalamer, zofran; ABI-007, acolbifen, actimmune, affinitak, aminopterin, arzoxifen, asoprisnil, atamestane, atrasentan, avastin, BAY 43-9006 (sorafenib), CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon-gamma, intron-PEG, ixabepilone, keyhole limpet hemocyanine, L-651582, lanreotide, lasofoxifen, libra, lonafarnib, miproxifen, minodronate, MS-209, liposomal MTP-PE, MX-6, nafarelin, nemorubicin, neovastat, nolatrexed, oblimersen, onko-TCS, osidem, paclitaxel polyglutamate, pamidronate disodium, PN-401, QS-21, quazepam, R-1549, raloxifen, ranpirnas, 13-cis-retic acid, satraplatin, seocalcitol, T-138067, tarceva, taxoprexin, thymosin-alpha-1, tiazofurin, tipifarnib, tirapazamine, TLK-286, toremifen, transMID-107R, valspodar, vapreotide, vatalanib, verteporfin, vinflunin, Z-100, zoledronic acid and combinations of the foregoing.

180. The construct of claim 175, wherein the affinity reagent is a protein comprising at least one unnatural amino acid, a protein in phage, or a therapeutic protein.

181. The construct of claim 175, wherein the affinity reagent is selected from the group consisting of designed ankyrin repeat proteins (DARPins), HEL4 Vh domains (Predator), Z-domain of staphylococcal protein A (Affibody), archeal “7 kDa DNA binder” protein family (Affitin), carbohydrate-binding module (CBD domain), cystine-knot miniprotein (knottin), fibronectin type III domain (monobody, Adnectin), γ-B-crystallin (Affilin), cystatins (Affimers), triple helix coiled coil domains (Alphabodies), lipocalin domains (Anticalins), A domains of various membrane receptors (Avimers), SH3 domains of Fyn (Fynomers), Kunitz domain peptides, and combinations thereof.

182. The construct of claim 172, wherein the linker is a polyoxazoline, polyacrylomorpholine, polyvinylpyrrolidone, polyphosphazene, polyethylene-co-maleic acid anhydride, polystyrene-co-maleic acid anhydride, poly(1-hydroxymethylethylene hydroxymethyl formal) (“PHF”), a polyhydroxyalkylacrylate, 2-methyacryloyloxy-2′-ethyltrimethylammonium phosphate (“MPC”), or a structure selected from: 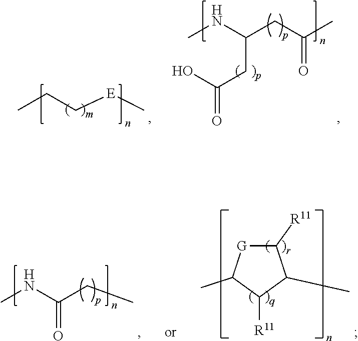
wherein:
m is 0-10;
n is 1-100;
each p independently is 0, 1, 2, 3, or 4;
q is 0, 1, or 2;
r is 1 or 2;
E is NH or CHR¹⁰;
G is O, CH₂, CHOH, CHNH₂, CHCOOH, or CHSO₃H;
R¹⁰ is OH, NH₂, or COOH;
each R¹¹ independently is H, OH, NH₂, or COOH.

183. A method comprising
(a) contacting a first fusion protein comprising a first reactive enzyme comprising cutinase with a linker comprising a functional group specific for irreversibly inhibiting the first reactive enzyme at a first terminus thereby coupling the first fusion protein and the linker at the first terminus, and
(b) contacting a second fusion protein comprising a second reactive enzyme comprising HaloTag or SnapTag with a second terminus of the linker, the second terminus of the linker comprising a functional group specific for irreversibly inhibiting the second reactive enzyme thereby coupling the second fusion protein and the linker at the second terminus.

184. The method of claim 183, wherein steps (a) and (b) are performed sequentially.

185. The method of claim 183, wherein steps (a) and (b) are preformed contemporaneously.

186. The method of claim 183, wherein the first fusion protein further comprises a first affinity reagent and the second fusion protein further comprises a second affinity reagent.

187. The method of claim 186, wherein the first and second affinity reagents are independently selected from the group consisting of antibody or fragment thereof, small molecule, monobody, protein, and combinations thereof.

188. The method of claim 186, wherein the first and/or second affinity reagent is selected from the group consisting of designed ankyrin repeat proteins (DARPins), HEL4 Vh domains (Predator), Z-domain of staphylococcal protein A (Affibody), archeal “7 kDa DNA binder” protein family (Affitin), carbohydrate-binding module (CBD domain), cystine-knot miniprotein (knottin), fibronectin type III domain (monobody, Adnectin), γ-B-crystallin (Affilin), cystatins (Affimers), triple helix coiled coil domains (Alphabodies), lipocalin domains (Anticalins), A domains of various membrane receptors (Avimers), SH3 domains of Fyn (Fynomers), Kunitz domain peptides, and combinations thereof.

189. The method of claim 183, wherein the linker is a of polyoxazoline, polyacrylomorpholine, polyvinylpyrrolidone, polyphosphazene, polyethylene-co-maleic acid anhydride, polystyrene-co-maleic acid anhydride, poly(1-hydroxymethylethylene hydroxymethyl formal) (“PHF”), a polyhydroxyalkylacrylate, 2-methyacryloyloxy-2′-ethyltrimethylammonium phosphate (“MPC”), or a structure selected from: 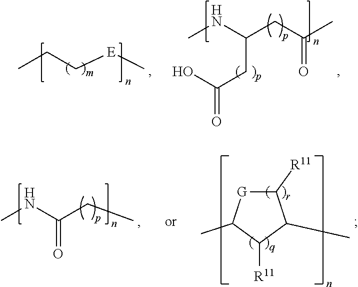
wherein:
m is 0-10;
n is 1-100;
each p independently is 0, 1, 2, 3, or 4;
q is 0, 1, or 2;
r is 1 or 2;
E is NH or CHR¹⁰;
G is O, CH₂, CHOH, CHNH₂, CHCOOH, or CHSO₃H;
R¹⁰ is OH, NH₂, or COOH;
each R¹¹ independently is H, OH, NH₂, or COOH.

190. A method comprising administering the construct of claim 172 to a patient in need thereof.

191. The method of claim 190, wherein the patient suffers from breast cancer, inhalational anthrax, rheumatoid arthritis, systemic juvenile idiopathic arthritis, Hodgkin lymphoma, systemic anaplastic large cell lymphoma, chronic lymphocytic leukemia, non-Hodgkin's lymphoma, diffuse large B cell lymphoma, multiple sclerosis, systemic lupus erythematosus, gastric or gastro-esophageal junction adenocarcinoma, metastatic non-small-cell lung carcinoma, ulcerative colitis, Crohn's disease, follicular lymphoma, melanoma, macular degeneration, osteoporosis, treatment-induced bone loss, metastases to bone, giant cell tumor of bone, malignant ascites, psoriatic arthritis, ankylosing spondylitis, metastatic renal cell cancer, prostate cancer, ovarian cancer, colorectal cancer, multiple myeloma, and Castleman's disease.


SEQUENCE LISTING (23 sequences)

SEQ ID NO: 1 - DNA, 22 bp, synthetic construct
  misc_feature 1..22: note = Synthetic Polynucleotide
  source 1..22: mol_type = other DNA
tttgcgtagtgtgtggtgcttt

SEQ ID NO: 2 - DNA, 22 bp, synthetic construct
  misc_feature 1..22: note = Synthetic Polynucleotide
  source 1..22: mol_type = other DNA
tttgcgtggggtgtggtgcttt

SEQ ID NO: 3 - DNA, 22 bp, synthetic construct
  misc_feature 1..22: note = Synthetic Polynucleotide
  source 1..22: mol_type = other DNA
tttgcgtagggtgtggtgcttt

SEQ ID NO: 4 - DNA, 44 bp, synthetic construct
  misc_feature 1..44: note = Synthetic Polynucleotide
  source 1..44: mol_type = other DNA
cgcgcaccgaaaggtgcgtattgtctatagcccagatttaagga

SEQ ID NO: 5 - DNA, 34 bp, synthetic construct
  misc_feature 1..34: note = Synthetic Polynucleotide
  source 1..34: mol_type = other DNA
ccatttctcgaagagaaaccggtaaatgcgccct

SEQ ID NO: 6 - DNA, 39 bp, synthetic construct
  misc_feature 1..39: note = Synthetic Polynucleotide
  source 1..39: mol_type = other DNA
cacacactttatgaatataaagtatagtgttatacttta

SEQ ID NO: 7 - DNA, 40 bp, synthetic construct
  misc_feature 1..40: note = Synthetic Polynucleotide
  source 1..40: mol_type = other DNA
acgtttctgaacgaagtgaagaaacgtctaagtgcgccct

SEQ ID NO: 8 - protein, 440 aa, synthetic construct
  REGION 1..440: note = Synthetic Polypeptide
MGLPTSNPAQELEARQLGRTTRDDLINGNSASCADVIFIYARGSTETGNLGTLGPSIASNLESAFGKDGVWIQGVGGAYRATLGDNALPRGTSSAAIREMLGLFQQANTKCPDATLIAGGYSQGAALAAASIEDLDSAIRDKIAGTVLFGYTKNLQNRGRIPNYPADRTKVFCNTGDLVCTGSLIVAAPHLAYGPDARGPAPEFLIEKVRAVRGSAEAAAKEAAAKDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 236 aa, synthetic construct
  REGION 1..236: note = Synthetic Polypeptide
MEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCLVPRGSHHHHHH

SEQ ID NO: 10 - protein, 406 aa, synthetic construct
  REGION 1..406: note = Synthetic Polypeptide
MDKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGEAAAKEAAAKDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 236 aa, synthetic construct; identical to SEQ ID NO: 9
  REGION 1..236: note = Synthetic Polypeptide

SEQ ID NO: 12 - protein, 440 aa, synthetic construct
  REGION 1..440: note = Synthetic Polypeptide
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEAAAKEAAAKGLPTSNPAQELEARQLGRTTRDDLINGNSASCADVIFIYARGSTETGNLGTLGPSIASNLESAFGKDGVWIQGVGGAYRATLGDNALPRGTSSAAIREMLGLFQQANTKCPDATLIAGGYSQGAALAAASIEDLDSAIRDKIAGTVLFGYTKNLQNRGRIPNYPADRTKVFCNTGDLVCTGSLIVAAPHLAYGPDARGPAPEFLIEKVRAVRGSA

SEQ ID NO: 13 - protein, 236 aa, synthetic construct; identical to SEQ ID NO: 9
  REGION 1..236: note = Synthetic Polypeptide

SEQ ID NO: 14 - protein, 406 aa, synthetic construct
  REGION 1..406: note = Synthetic Polypeptide
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEAAAKEAAAKDKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLG

SEQ ID NO: 15 - protein, 236 aa, synthetic construct; identical to SEQ ID NO: 9
  REGION 1..236: note = Synthetic Polypeptide

SEQ ID NO: 16 - protein, 215 aa, synthetic construct
  REGION 1..215: note = Synthetic Polypeptide
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 461 aa, synthetic construct
  REGION 1..461: note = Synthetic Polypeptide
MGLPTSNPAQELEARQLGRTTRDDLINGNSASCADVIFIYARGSTETGNLGTLGPSIASNLESAFGKDGVWIQGVGGAYRATLGDNALPRGTSSAAIREMLGLFQQANTKCPDATLIAGGYSQGAALAAASIEDLDSAIRDKIAGTVLFGYTKNLQNRGRIPNYPADRTKVFCNTGDLVCTGSLIVAAPHLAYGPDARGPAPEFLIEKVRAVRGSAEAAAKEAAAKEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCLVPRGSHHHHHH

SEQ ID NO: 18 - protein, 215 aa, synthetic construct; identical to SEQ ID NO: 16
  REGION 1..215: note = Synthetic Polypeptide

SEQ ID NO: 19 - protein, 427 aa, synthetic construct
  REGION 1..427: note = Synthetic Polypeptide
MDKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGEAAAKEAAAKEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCLVPRGSHHHHHH

SEQ ID NO: 20 - protein, 215 aa, synthetic construct; identical to SEQ ID NO: 16
  REGION 1..215: note = Synthetic Polypeptide

SEQ ID NO: 21 - protein, 461 aa, synthetic construct
  REGION 1..461: note = Synthetic Polypeptide
MEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEAAAKEAAAKGLPTSNPAQELEARQLGRTTRDDLINGNSASCADVIFIYARGSTETGNLGTLGPSIASNLESAFGKDGVWIQGVGGAYRATLGDNALPRGTSSAAIREMLGLFQQANTKCPDATLIAGGYSQGAALAAASIEDLDSAIRDKIAGTVLFGYTKNLQNRGRIPNYPADRTKVFCNTGDLVCTGSLIVAAPHLAYGPDARGPAPEFLIEKVRAVRGSALVPRGSHHHHHH

SEQ ID NO: 22 - protein, 215 aa, synthetic construct; identical to SEQ ID NO: 16
  REGION 1..215: note = Synthetic Polypeptide

SEQ ID NO: 23 - protein, 427 aa, synthetic construct
  REGION 1..427: note = Synthetic Polypeptide
MEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEAAAKEAAAKDKDCEMKRTTLDSPLGKLELSGCEQGLHEIIFLGKGTSAADAVEVPAPAAVLGGPEPLMQATAWLNAYFHQPEAIEEFPVPALHHPVFQQESFTRQVLWKLLKVVKFGEVISYSHLAALAGNPAATAAVKTALSGNPVPILIPCHRVVQGDLDVGGYEGGLAVKEWLLAHEGHRLGKPGLGLVPRGSHHHHHH